(19) United States
(12) Patent Application Publication
(10) Pub. No.: US 2019/0125937 A1
(43) Pub. Date: May 2, 2019

(54) Implantable Device for Implantation of Cells Having Anti-Inflammatory and Vascularization Capabilities and Methods of Making Thereof

(71) Applicant: Beta-O2 Technologies Ltd., Rosh-Haayin (IL)

(72) Inventors: Avi Rotem, Petach-Tikva (IL); Baruch Zimmerman, Rosh-Haayin (IL); Uriel Barkai, Rosh-Haayin (IL); Shiri Maimon, Rosh-Haayin (IL)

(21) Appl. No.: 16/097,138

(22) PCT Filed: Apr. 4, 2017
(86) PCT No.: PCT/IB2017/000461

§ 371 Date: Oct. 26, 2018

Related U.S. Application Data
(60) Provisional application No. 62/317,990, filed on Apr. 4, 2016.

(51) Int. Cl. A61L 31/14; B01D 69/12; A61L 31/00; C12N 5/071; C12N 5/0735; C12N 5/00; A61L 31/16

(52) U.S. Cl. CPC A61L 31/146; B01D 69/125; A61L 31/005; C12N 5/0614; B01D 2323/40; C12N 5/0018; A61L 31/16; B01D 2323/345; B01D 2325/36; C12N 5/0606

(57) ABSTRACT

A method includes spreading a solution including a polyether and a photoinitiator onto a hydrophilic porous membrane, impregnating hydrophilic the porous membrane with the solution, and curing the solution located within the hydrophilic porous membrane by exposure to ultraviolet light to produce a composite membrane.

### CROSS REFERENCE TO RELATED APPLICATIONS

This application claims priority to U.S. Provisional Patent Application No. 62/317,990, filed on Apr. 4, 2016, the contents of which are incorporated herein by reference in their entirety.

### FIELD OF THE INVENTION

The exemplary embodiments relate to an implantable medical device having a unit that comprises transplanted cells protected from the host immune system by a membrane impregnated with hydrogel and oxygen unit that may be separated, and to methods for making such devices.

### BACKGROUND

Several disorders arising from hyposecretion of one or more substances such as hormones are known.

Disorders arising from hyposecretion of a hormone are usually treated by administration of the missing hormone. However, despite advances in understanding and treating many of these diseases, it is often not possible to precisely regulate metabolism with exogenous hormones.

Organ transplantation is often not a viable treatment for most of these disorders for several reasons, including, for example, rejection of the transplanted tissue by the immune system. Isolated cells may be implanted in the body after being treated to prevent rejection e.g. by immunosuppression, radiation or encapsulation. The encapsulating material is selected so as to be biocompatible and to allow diffusion of small molecules between the cells and the environment while shielding the cells from immunoglobulins and cells of the immune system.

Encapsulated β-cells or islets of Langerhans (the tissue producing the insulin), for example, can be injected into the portal vein or embedded under the skin, in the abdominal cavity, or transplanted in other locations. The success of many cellular transplants is compromised not only due to graft-host rejections, but also on account of ischemic conditions generated by insufficient oxygen supply to the transplant. Oxygen is vital for the physiological processes and functionality of the implanted cells. An insufficient supply of oxygen to the implanted cells, often leads to cell loss of functionality or death. Oxygen provision is a vital component in sustaining the viability and functionality of transplanted cells.

### SUMMARY

In one embodiment, a method includes spreading a solution on a hydrophilic porous membrane, the solution including a polyether and a photoinitiator, impregnating the hydrophilic porous membrane with the solution, and curing the solution located within the hydrophilic porous membrane by exposure to ultraviolet light to produce a composite membrane.

In one embodiment, the composite membrane has a suitable pore size such that molecules having a molecular weight of greater than about 100,000 Daltons are prevented from passing through the membrane.

In one embodiment, the method also includes drying the composite membrane by either oven-drying or lyophilizing. In one embodiment, the method also includes placing the dried composite membrane in an implantable device.

In one embodiment, the method also includes, prior to the step of placing the dried composite membrane in the implantable device, performing the steps of mixing functional cells with a polymer to produce a cell mixture, placing the cell mixture on the composite membrane, and cross-linking the cell mixture with a cross-linking agent to produce an embedded cell layer adjacent the composite membrane, wherein, when the dried composite membrane is placed in the implantable device, the embedded cell layer is also placed in the implantable device. In one embodiment, the cross-linking agent includes at least one of barium, strontium, and calcium. In one embodiment, the functional cells include at least one of islets of Langerhans, stem cells, and adrenal cells.

In one embodiment, the implantable device is configured to receive a supply of oxygen from an external source. In one embodiment, the composite membrane and the embedded cell layer are positioned in the implantable device such that the composite membrane is positioned between the external oxygen source and the embedded cell layer.

In one embodiment, the method also includes culturing functional cells in a basal medium and injecting the cultured functional cells and the basal medium into a tissue chamber of the implantable device. In one embodiment, the functional cells include at least one of islets of Langerhans, stem cells, and adrenal cells. In one embodiment, the implantable device is configured to receive a supply of oxygen from an external source. In one embodiment, the composite membrane and the tissue chamber are positioned in the implantable device such that, when the implantable device is implanted within a host, the composite membrane is positioned between the tissue chamber and tissue of the host.

In one embodiment, the polyether includes at least one of polyethylene glycol diacrylate, polyethylene glycol acrylate, and polyethylene glycol dimethacrylate. In one embodiment, the step of impregnating the hydrophilic porous membrane with the solution includes compressing the hydrophilic porous membrane and the solution between two pieces of a transparent material to impregnate the hydrophilic porous membrane with the solution, and wherein the curing step is performed while the hydrophilic porous membrane and the solution are compressed between the two pieces of the transparent material.

In one embodiment, a method includes placing an HM alginate solution on a hydrophilic porous membrane, exposing the HM alginate solution on the hydrophilic porous membrane to a vacuum pressure to produce a hydrophilic porous membrane impregnated with HM alginate, exposing the hydrophilic porous membrane impregnated with HM alginate to a cross-linking solution to cross-link the HM alginate and produce a hydrophilic porous membrane impregnated with cross-linked HM alginate, and lyophilizing the hydrophilic porous membrane impregnated with cross-linked HM alginate to produce a composite membrane.

In one embodiment, the cross-linking solution includes at least one of strontium, barium, and calcium. In one embodiment, the method also includes mixing functional cells with HG-alginate to produce a cell mixture, placing the cell mixture on the composite membrane, and cross-linking the cell mixture with a cross-linking agent to produce an embedded cell layer adjacent to the composite membrane.

In one embodiment, the cross-linking agent includes at least one of strontium, barium, and calcium. In one embodiment, the method also includes installing the composite membrane and the embedded cell layer in an implantable device that is configured to receive oxygen from an external oxygen source, wherein the composite membrane and the embedded cell layer are positioned in the implantable device such that the composite membrane is positioned between the external oxygen source and the embedded cell layer. In one embodiment, the functional cells include at least one of islets of Langerhans, stem cells, and adrenal cells. I

In one embodiment, the method also includes immobilizing biologically active molecules in the HG-alginate. In one embodiment, the biologically active molecules include at least one of anti-inflammatory molecules and anti-apoptotic drugs.

In one embodiment, the present invention provides an implantable medical system, comprising:
a. a gas unit for supplying gas, wherein said gas comprises essentially oxygen; and
b. at least one of a unit containing a plurality of functional cells, the unit having a certain degree of physical flexibility and configured to receive oxygen from the gas unit so as to maintain the functional cells in a viable and functional condition.

In one embodiment, the system further comprises at least one distributor configured to distribute the gas from the gas unit to the at least one of a unit containing a plurality of functional cells.

In one embodiment, the gas unit is a pressurized reservoir of gas that can be replenished through a subcutaneous implantable port and wherein the port is configured to receive gas through a needle adapted to penetrate the replenishing port.

In one embodiment, when the implantable medical system is implanted in an animal, the port is further configured to receive gas through a needle adapted to penetrate the replenishing port and the skin of the animal.

In one embodiment, a uni-directional valve is provided between the replenishing port and the gas unit.

In one embodiment, the uni-directional valve is configured to transfer gas from the port to the gas unit.

In one embodiment, the gas unit is an oxygen generator.

In one embodiment, the oxygen generator comprises electrodes that produce oxygen by electrolysis.

In one embodiment, the oxygen generator generates oxygen by hydrolysis and comprises a pair of electrodes and a power source.

### BRIEF DESCRIPTION OF THE DRAWINGS

FIG. 1 illustrates an embodiment of the apparatus of the present invention. (1) Body of the device containing the gas unit. (2) Cover of the device containing the composite dry membrane (9). (3) Tube connecting the gas chamber with the ports (4).

FIG. 2 illustrates an embodiment of the apparatus of the present invention, shown without the cover. (5) Body of the device containing the gas unit. (6) Gas permeable membrane. (7) O-Ring sealing. (12) Holes for the cover.

FIGS. 3A and 3B illustrate an embodiment of the apparatus of the present invention, showing the cover of the apparatus. FIG. 3A: (7) holes for needle. (9) Composite membrane FIG. 3B: (8) silicon rubber plugs. (10) Composite membrane under the grove for the cells or tissue.

FIG. 4 illustrates an embodiment of the apparatus of the present invention, showing the closure of the apparatus. Cover (2) loaded with gel mix with tissue or cells (10) are closed on the body (1). O-ring (7) prevents body liquid to penetrate the device.

FIG. 5A shows a top view of the apparatus with hydrophilic porous polytetrafluoroethylene (“PTFE”) (Biopore) membrane glued on a plastic frame of the implanted device. FIG. 5B shows 4-8% HM alginate placed on the Biopore membrane.

FIG. 6A shows the PTFE (Biopore) membrane according to some embodiments of the present invention as is and FIG. 6B shows the Biopore membrane according to some embodiments of the present invention impregnated with HM alginate.

FIG. 7 shows a schematic overview of the integration process according to some embodiments of the present invention (drawing not to scale): (i) Strontium cross-linked HG alginate containing the islets graft and (ii) Barium cross-linked HM alginate reinforced by hydrophilic PTFE membrane.

FIG. 8 A shows the inside of an implantable device with the membrane-alginate structure according to some embodiments of the present invention. FIG. 8 B is a close up of FIG. 8 A.

FIGS. 9 A and B shows an alginate membrane containing functional cells that has been removed from a device according to some embodiments of the present invention, after the device had been implanted in a human for 10 months. Tissue is stained with dithizone, which binds to insulin, demonstrating the viability of the tissue.

FIG. 10 shows the position of an implanted system according to some embodiments of the present invention.

FIG. 11 shows a schematic overview of a process for impregnating a Biopore membrane according to some embodiments of the present invention.

FIG. 12A shows a portion of an implantable device according to some embodiments of the present invention. FIG. 12B shows a cross-sectional view of the implantable device of FIG. 12A along the cross-section indicated in FIG. 12A.

FIG. 13 shows an illustration of an experimental injection system according to some embodiments of the present invention.

FIG. 14 shows photographs of various views of the experimental injection system of FIG. 13.

FIG. 15 shows an illustration of an experimental injection similar similar to that of FIG. 13.

FIG. 16 shows an illustration of an experimental injection system similar to that of FIGS. 14 and 15.

FIG. 17 shows photographs of various views of an experimental injection system similar to that shown in FIG. 15, as used to inject mouse insulinoma cells.

FIG. 18 shows a cross-sectional view of a portion of an implantable device including a composite membrane and a tissue compartment.

### DETAILED DESCRIPTION

For clarity of disclosure, and not by way of limitation, the detailed description of the invention is divided into the following subsections that describe or illustrate certain features, embodiments or applications of the present invention.

Throughout the specification and claims, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

In some embodiments, the present invention provides an implantable medical system, comprising:
a. a gas unit for supplying gas, wherein said gas comprises essentially oxygen; and
b. at least one of a unit containing a plurality of functional cells, the unit having a certain degree of physical flexibility and configured to receive oxygen from the gas unit so as to maintain the functional cells in a viable and functional condition.

In some embodiments, the system further comprises at least one distributor configured to distribute the gas from the gas unit to the at least one of a unit containing a plurality of functional cells.

In some embodiments, the gas unit is a pressurized reservoir of gas that can be replenished through a subcutaneous implantable port and wherein the port is configured to receive gas through a needle adapted to penetrate the replenishing port.

In some embodiments, when the implantable medical system is implanted in an animal, the port is further configured to receive gas through a needle adapted to penetrate the replenishing port and the skin of the animal.

In some embodiments, a uni-directional valve is provided between the replenishing port and the gas unit.

In some embodiments, the uni-directional valve is configured to transfer gas from the port to the gas unit.

In one embodiment, the gas unit is an oxygen generator.

In one embodiment, the oxygen generator comprises electrodes that produce oxygen by electrolysis.

In one embodiment, the oxygen generator generates oxygen by hydrolysis and comprises a pair of electrodes and a power source.

In some embodiments, the implantable medical device comprises a gas mixture comprising oxygen at a concentration of between 40% and 95% and balance of nitrogen. In some embodiments, the oxygen mixture comprises 5% carbon dioxide. In some embodiments, the pressure of the gas mixture in the gas chamber is between 1.0 atm (ambient pressure) and 10 atm. In some embodiments, the pressure of the gas mixture in the gas chamber is between 5.0 atm (ambient pressure) and 10 atm. In some embodiments, the pressure of the gas mixture in the gas chamber is between 1.0 atmosphere (atm) (ambient pressure) and 5 atm. In some embodiments, the source of oxygen (i.e., generator) comprises around 5% carbon dioxide in order to maintain a balance of concentrations of carbon dioxide between the inside of the housing and the body.

Furthermore and in accordance with an embodiment, the functional cells are selected from a group comprising islets of Langerhans, adrenal cells, stem cells that were matured to be beta cells or alpha cells and genetic implantable cells.

Furthermore and in accordance with an embodiment, at least one of a unit containing a plurality of functional cells comprises opposite positioned compartments of substantially the same dimensions, both compartments are provided with a relatively high surface area face through which oxygen can diffuse and reach the functional cells inside the unit.

In some embodiments, an apparatus can have the dimensions of 32 mm in diameter and 9 mm wide. In some embodiments, an apparatus can have the dimensions of between 10 mm-50 mm in diameter and/or 1 mm-20 mm wide. In some embodiments, an apparatus can have the dimensions of between 10 mm-40 mm in diameter. In some embodiments, an apparatus can have the dimensions of between 10 mm-30 mm in diameter. In some embodiments, an apparatus can have the dimensions of between 10 mm-20 mm in diameter. In some embodiments, an apparatus can have the dimensions of between 20 mm-50 mm in diameter. In some embodiments, an apparatus can have the dimensions of between 30 mm-50 mm in diameter. In some embodiments, an apparatus can have the dimensions of between 40 mm-50 mm in diameter. In some embodiments, an apparatus can have the dimensions of between 1 mm-15 mm in width. In some embodiments, an apparatus can have the dimensions of between 1 mm-10 mm in width. In some embodiments, an apparatus can have the dimensions of between 1 mm-5 mm in width. In some embodiments, an apparatus can have the dimensions of between 5 mm-20 mm in width. In some embodiments, an apparatus can have the dimensions of between 10 mm-20 mm in width. In some embodiments, an apparatus can have the dimensions of between 15 mm-20 mm in width.

Furthermore and in accordance with an embodiment, the high surface area face is covered with a layer that facilitates transfer of oxygen.

Furthermore and in accordance with an embodiment, said layer is a silicone layer.

Furthermore and in accordance with an embodiment, outer sides of at least one of a unit containing a plurality of functional cells is covered with another layer permeable to nutrients and bio-materials that may be produced by the functional cells and impermeable to immunologic factors.

Furthermore and in accordance with an embodiment, the at least one of a unit containing a plurality of functional cells are disc-like and are having a thickness of about 20-2,000 μm.

Furthermore and in accordance with an embodiment, the functional cells are embedded in a matrix within the at least one of a unit containing a plurality of functional cells.

Furthermore and in accordance with an embodiment, the matrix is made of materials selected from a group that comprises hydrogels (e.g. hydrogels based on polyethylene glycol (“PEG”), other PEG-based polymers such as PEG-diacrylate (“PEG-DA”) or PEG-dimethacrylate (“PEG-DMA”), PEG-acrylate (“PEG-A”), alginate, collagen, and combination thereof).

Furthermore and in accordance with an embodiment, the functional cells in the at least one of a unit containing a plurality of functional cells are trapped within a porous structure.

Furthermore and in accordance with an embodiment, the at least one of a unit containing a plurality of functional cells comprises a plurality of subunits having substantially large surface area that allows transfer of oxygen wherein each subunit is provided with functional cells embedded in a matrix.

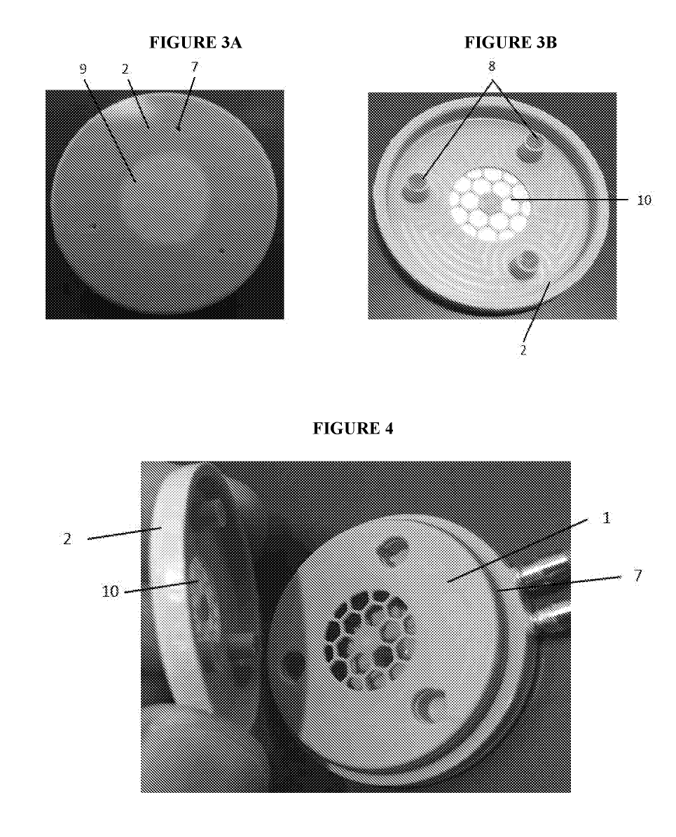

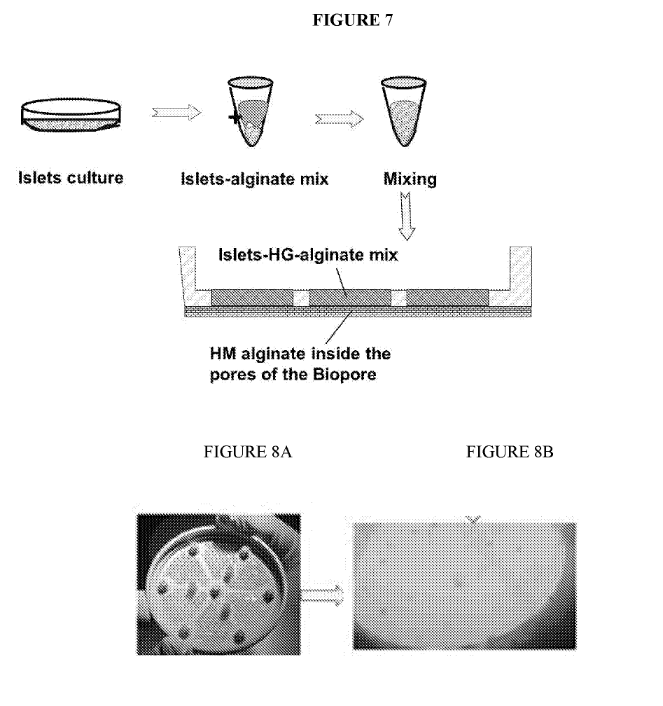

As illustrated in FIGS. 3 and 7, to integrate the cells and/or tissue (e.g. islets of Langerhans), the cells and/or tissue are mixed with 3.5% HG alginate, located in the grove (10) inside of the cover (2) and cross-linked with strontium for 22 min. In some embodiments, the cross-linking can be performed in e.g., but not limited to, 1-60 minutes. In some embodiments, the cross-linking can be performed in e.g., but not limited to, 5-35 minutes. In some embodiments, the cross-linking can be performed in e.g., but not limited to, 5-25 minutes. In some embodiments, the cross-linking can be performed in e.g., but not limited to, 5-15 minutes. In some embodiments, the cross-linking can be performed in e.g., but not limited to, 15-35 minutes. In some embodiments, the cross-linking can be performed in e.g., but not limited to, 25-35 minutes.

Furthermore and in accordance with an embodiment, said subunits are arranged similarly to an egg carton, wherein the diameter of each subunit is about 10-2,500 μm. In some embodiments, each subunit is about 10-2,000 μm. In some embodiments, each subunit is about 10-1,500 μm. In some embodiments, each subunit is about 10-1,000 μm. In some embodiments, each subunit is about 10-500 μm. In some embodiments, each subunit is about 10-100 μm. In some embodiments, each subunit is about 100-2,500 μm. In some embodiments, each subunit is about 500-2,500 μm. In some embodiments, each subunit is about 1,000-2,500 μm. In some embodiments, each subunit is about 1,500-2,500 μm. In some embodiments, each subunit is about 2,000-2,500 μm.

Furthermore and in accordance with an embodiment, the at least one of a unit containing a plurality of functional cells is provided with inner projections configured to allow the functional cells to be captured.

Further and in accordance with an embodiment, the apparatus is configured to maintain the viability and functionality of cells or tissue embedded in the matrix by (i) continuously and actively supplying oxygen and (ii) protecting the cells or tissue embedded in the matrix from the host immune system by use of a composite membrane configured to permit small dissolved molecules (e.g., but not limited to, glucose and insulin) to enter the apparatus and prevent the transfer of large water soluble molecules configured to initiate or propagate an immune response, such as, but not limited to, immunoglobulins, complement components, etc., to pass through the membrane. In some embodiments, the apparatus can be used for living tissue or cells, where the living tissue or cells comprise: islets of Langerhans and adrenal cells.

The present invention relates generally to an implantable medical device having a layer or layers of transplanted cells or transplanted tissue, where the layer of cells or tissue can include Islets of Langerhans and/or adrenal cells, where the layer of cells is embedded in a hydrogel, where the hydrogel is HG alginate, and where the layer of cells is supplied with oxygen delivered from an internal or external gas chamber or oxygen generator, where a gas permeable membrane separates the oxygen tank and the tissue.

In some embodiments, the gas permeable membrane comprises a gas permeable material, where the gas permeable material is silicone rubber or silicone PTFE, and where the gas permeable material has a thickness of between 10 μm and 2000 μm. In some embodiments, the gas permeable material has a thickness of between 100 μm and 2000 μm. In some embodiments, the gas permeable material has a thickness of between 500 μm and 2000 μm. In some embodiments, the gas permeable material has a thickness of between 1000 μm and 2000 μm. In some embodiments, the gas permeable material has a thickness of between 1500 μm and 2000 μm. In some embodiments, the gas permeable material has a thickness of between 10 μm and 1500 μm. In some embodiments, the gas permeable material has a thickness of between 10 μm and 1000 μm. In some embodiments, the gas permeable material has a thickness of between 10 μm and 500 μm. In some embodiments, the gas permeable material has a thickness of between 10 μm and 100 μm.

In some embodiments, the oxygen gas passes from the gas unit through the gas permeable membrane, dissolves in the hydrogel containing-tissue and diffuses to the embedded tissue or cells. In some embodiments, the gas mixture is loaded through one, two or more implantable ports, connected to the gas unit by tubes. In some embodiments, the gas mixture is replenished every period, e.g. every 24 hours or every few days. In some embodiments, the tissue or cells are separated from bodily liquids by a composite membrane, where the composite membrane is configured to allow the transfer of small water soluble molecules (e.g., but not limited to, glucose and insulin), and prevents and/or reduces the transfer of large water soluble molecules configured to illicit an immune response, (e.g., but not limited to, immunoglobulins, complement components). In some embodiments, the composite membrane comprises a porous hydrophilic membrane (e.g., but not limited to, a PTFE hydrophilic membrane) configured for use as a scaffold and the porous hydrophilic membrane's void volume containing a hydrogel and/or an alginate (e.g., but not limited to, PEG, PEG-DA, PEG-DMA, PEG-A, HM alginate) as a filler, where the alginate is cross-linked with a divalent ion (e.g., but not limited to, through the use of barium, strontium, calcium, or another cross-linking agent). In some embodiments, the composite membrane is dried (e.g., but not limited to, freeze-dried) before device integration. In some embodiments, the composite membrane is sterilized by incubation in ethylene oxide at a temperature between 32 and 36 deg C. In some embodiments, the freeze-dried composite membrane can be stored at 4 deg C. or 25 deg C. In some embodiments, the composite membrane is attached to the cover by glue or by a mechanical attachment. In some embodiments, the gel encapsulating the islets is HG alginate, HM alginate, another hydrogel, or a combination thereof, and the gel is configured for immobilization of biologically active molecules. In some embodiments, the biologically active molecules are anti-inflammatory molecules, where the anti-inflammatory molecules are prostaglandins, leukotrienes, adenosine, or any combination thereof. In some embodiments, the biologically active molecules are anti-apoptotic drugs, where the anti-apoptotic drugs are caspase inhibitors. In some embodiments, the biologically active molecules are hormones comprising IGF2, GLP-1, or any combination thereof. In some embodiments, the biologically active molecules induce angiogenesis and comprise VEGF.

The present invention relates generally to a process, in which the filler, where the filler is HM alginate, is introduced into the porous membrane, where the porous membrane is PTFE hydrophilic membrane, under vacuum conditions of between 0.01 mbar and 0.9 mbar. In some embodiments, the vacuum conditions are between 0.1 and 0.9 bar. In some embodiments, the vacuum conditions are between 0.5 and 0.9 bar. In some embodiments, the vacuum conditions are between 0.01 and 0.5 bar. In some embodiments, the vacuum conditions are between 0.01 and 0.1 bar.

In some embodiments, the cells or tissue derived from the islets of Langerhans is positioned inside a cover in a thin layer measures between 100 μm-5000 μm in thickness. In some embodiments, the thin layer measures between 500 μm-5000 μm in thickness. In some embodiments, the thin layer measures between 1000 μm-5000 μm in thickness. In some embodiments, the thin layer measures between 2500 μm-5000 μm in thickness. In some embodiments, the thin layer measures between 200 μm-2500 μm in thickness. In some embodiments, the thin layer measures between 200 μm-1000 μm in thickness. In some embodiments, the thin layer measures between 200 μm-500 μm in thickness. In some embodiments, the alginate is cross-linked with at least one divalent ion comprising barium, strontium, calcium, or any combination thereof. In some embodiments, the cover containing the cells or tissue, where the cells or tissue is derived from the Islets of Langerhans, further comprises silicon plugs. In some embodiments, isolated islets of Langerhans are layered on top of hydrogel (e.g. HG alginate) and pushed into the hydrogel using centrifuge at low g force of between 50 g and 500 g. In some embodiments, as a result the water adsorbed to the surface of the islets remains in the top layer, living the islets with minimal water around the islets. In some embodiments, the water on top is discarded and the islets are gently mixed with the hydrogel. In some embodiments, the mixture of the islets and hydrogel are layered in the cover of the device and the hydrogel (e.g. alginate) is cross-linked with divalent ion (e.g. strontium or barium). In some embodiments, the cover and the islets immobilized in the cross-linked hydrogel are placed on the body of the device containing the oxygen tank. In some embodiments, during the closure of the cover on the device, the air locked between the cover and the device is cleared by needle (e.g. 24 G) stuck in the silicon plugs in the cover. In some embodiments, the cover is closed on the body of the device. In some embodiments, a needle, for example, but not limited to, a G24 Huber needle, is inserted through the silicon plugs. In some embodiments, the cover is closed on low

In some embodiments, isolated islets derived from a pancreas obtain an oxygen supply by diffusion from the external environment, where oxygen diffuses radially inward from the islet surface and the oxygen is consumed by the cells so as to decrease the oxygen concentration toward the center of the islet. As an exemplary embodiment, for a spherical Islet Equivalent (IEQ) of human origin, containing an average of 1,560 cells and having a diameter of 150 μm, the outer islet surface has an oxygen partial pressure about 45-50 mmHg so as to allow the maintenance of the functionality of the cells.

In some embodiments of the apparatus of the present invention, the cells or tissue in the apparatus are maintained in a pH 7.3-7.4 environment by supplying the gas chamber with 5% CO₂. In some embodiments, the CO₂ partial pressure of tissue measures about 40 mm Hg.

In some embodiments, an inner alginate layer has a guluronic acid concentration of between 50% and 99%, e.g., between 67% and 71%. The multi-layer immune barrier further comprises a second, outer alginate structure that surrounds at least in part a first, an inner alginate structure. The second alginate structure has a mannuronic acid concentration of between 50% and 99%, e.g., between 54% and 58%. In some embodiments, the guluronic acid concentration ranges between 60% and 99%. In some embodiments, the guluronic acid concentration ranges between 70% and 99%. In some embodiments, the guluronic acid concentration ranges between 80% and 99%. In some embodiments, the guluronic acid concentration ranges between 90% and 99%. In some embodiments, the guluronic acid concentration ranges between 50% and 90%. In some embodiments, the guluronic acid concentration ranges between 50% and 80%. In some embodiments, the guluronic acid concentration ranges between 50% and 70%. In some embodiments, the guluronic acid concentration ranges between 50% and 60%. In some embodiments, the mannuronic acid concentration ranges between 60% and 99%. In some embodiments, the mannuronic acid concentration ranges between 70% and 99%. In some embodiments, the mannuronic acid concentration ranges between 80% and 99%. In some embodiments, the mannuronic acid concentration ranges between 90% and 99%. In some embodiments, the mannuronic acid concentration ranges between 50% and 90%. In some embodiments, the mannuronic acid concentration ranges between 50% and 80%. In some embodiments, the mannuronic acid concentration ranges between 50% and 70%. In some embodiments, the mannuronic acid concentration ranges between 50% and 60%.

In some embodiments, bioactive molecules, e.g., but not limited to, anti-inflammatory, anti-apoptotic, pro-angiogenic, or any combination thereof, are incorporated into at least one one of the hydrogel macrostructures. In some embodiments, the bioactive molecules can be integrated as molecules dissolved in the hydrogel, covalently bound, or following prior encapsulation into nanocapsules, liposomes, or dendrimers.

In some embodiments of the apparatus of the present invention, the apparatus comprises anti-inflammatory drugs, for example, but not limited to, compounds which act as inhibitor of prostaglandins and/or leukotrienes. In some embodiments, an inhibitor of prostaglandin and lipoxygenase controlling leukotriene synthesis, e.g., COX-2 inhibitors (e.g., non-steroidal anti-inflammatory drugs (NSAID)) increase cell survival and function by between 10-99% (e.g., but not limited to, 10%, 20%, 30%, 40%, 50%, 60%, 70%, 80%, 90%, etc.). In some embodiments, selective COX-2 inhibitors (FDA-approved (FDA-AD)) improve islets survival and function by between 10-99% (e.g., but not limited to, 10%, 20%, 30%, 40%, 50%, 60%, 70%, 80%, 90%, etc.). In some embodiments, COX-2 siRNA (FDA-not approved (FDA-NAD)) improves islets survival and function by between 10-99% (e.g., but not limited to, 10%, 20%, 30%, 40%, 50%, 60%, 70%, 80%, 90%, etc.). As an exemplary embodiment, 3 mg Ketoprofen (FDA-AD) or 3 mg diclofenac encapsulated in a hydrogel reduce the inflammatory response (e.g., but not limited to, 0.05%, 0.5%, etc. reduction in inflammatory response) and the degree of cell overgrowth over implanted micro-capsules (e.g., but not limited to, 0.05%, 0.5%, etc. cell overgrowth over implanted micro-capsules). In an exemplary embodiment, a 12-lipoxygenase (12LO) inhibitor (e.g., esculetin, gossypol, ferulic acid, ETYA, ethyl 3,4-dihydroxybenzylidenecyanoacetate, caffeic acid, baicalein, hinokitiol, ETI, 8, 11, 14-eicosatriynoic acid, 2-TEDC, CDC, 15(S)-HETrE, 3,4-dihydroxyphenyl ethanol, or any combination thereof (12LO inhibitors available at ChemCruz Biochemicals)) is used to reduce 12LO enzyme activity in isolated cells by between, e.g., but not limited to, 10-99% reduced 12LO enzyme activity.

In some embodiments of the apparatus of the present invention, the apparatus comprises anti-inflammatory drugs, for example, but not limited, to interleukin beta (IL-β) inhibitors such as Kineret, an anti-IL1b receptor antagonists (e.g., but not limited to, Kineret, which is a candidate drug approved for human use dosed at 1 mg/device, Efaroxan (FAD-NAD), adenosine (e.g., but not limited to a single dose of 5 mg/kg), or any combination thereof.

In some embodiments of the apparatus of the present invention, the apparatus comprises anti-apoptotic drugs, for example, but not limited to, inhibitors of cytokines (e.g., but not limited to, Tumor necrosis factor (TNF)-alpha, IL-1beta, interferon-gamma, etc., or any combination thereof), inhibitors of BCL-2 and/or Bc1-xL proteins (e.g., but not limited to BAD, Bax, etc., or any combination thereof), or any combination thereof.

In some embodiments of the apparatus of the present invention, the apparatus comprises anti-apoptotic drugs, for example, but not limited to, inhibitors of caspases, for example, but not limited to, pentapeptide V5 and/or 20 mg dehydroxymethylepoxyquinomicin (DHMEQ), 100 mg enricasan (Contatus pharmaceuticals), or any combination thereof.

In some embodiments of the apparatus of the present invention, the apparatus comprises anti-apoptotic drugs, for example, but not limited to hormones, for example, but not limited to, 10 mg IGF2, GLP-1, or any combination thereof, such as 30 mg Albiglutide (GlaxoSmithKline), 5 mg Bydureon (AstraZeneca), or any combination thereof.

In some embodiments of the apparatus of the present invention, the apparatus comprises anti-apoptotic drugs, for example, but not limited to, 1 gr curcumin.

In some embodiments of the apparatus of the present invention, the apparatus comprises anti-apoptotic drugs, for example, but not limited to, inhibitors of oxidative stress/reactive oxygen species (ROS), for example, but not limited to, antioxidants (e.g., but not limited to, glutamine, pyruvate, superoxide dismutase mimetric molecule, vitamin E, soluble vitamin E derivative, taurine and N-acetylcystein, omega-3 derivatives, 300 mg Ladostigin (Avraham Pharmaceuticals) or any combination thereof). In some embodiments, 500 mg Alpha-1 antitrypsin (AAT-FDA-AD) is included in the apparatus of the present invention.

In some embodiments of the apparatus of the present invention, the apparatus comprises pro-angiogenic factors, where the pro-angiogenic factors are: (i) immobilized on a scaffold and/or on a membrane, so as to result in a slow release of the pro-angiogenic factors at a predetermined concentration at site of implantation that increases the generation of blood vessels (e.g., but not limited to, an increase of 10%, 20%, 30%, 40%, 50%, 60%, 70%, 80%, 90%, 99%), and (ii) including platelets and/or platelet derivatives in the encapsulating scaffold, so as to result in an increase of platelet-derived micro-particles by, but not limited to, a 10-99% increase (e.g., but not limited to, 10%, 20%, 30%, 40%, 50%, 60%, 70%, 80%, 90%, etc.). In some embodiments, the slow release occurs over a period of about two weeks. In some embodiments, the slow release occurs over a period of greater than two weeks. In some embodiments, the slow release occurs over a period that is sufficiently long such that extends for the duration of inflammation that occurs after implantation.

Reference is now made to the following examples, which together with the above descriptions illustrate some embodiments of the invention in a non-limiting fashion.

### EXAMPLES

### Example 1: Process Description

A gas permeable membrane, e.g. 25 μm silicon PTFE (Silon), was attached (e.g., but not limited to, glued) on the body (1) of the device using medical silicon glue (e.g., but not limited to, MED2000, Neusil), as shown in FIG. 2, item 6. In some embodiments, the membrane has a thickness of between 25 μm and 75 μm.

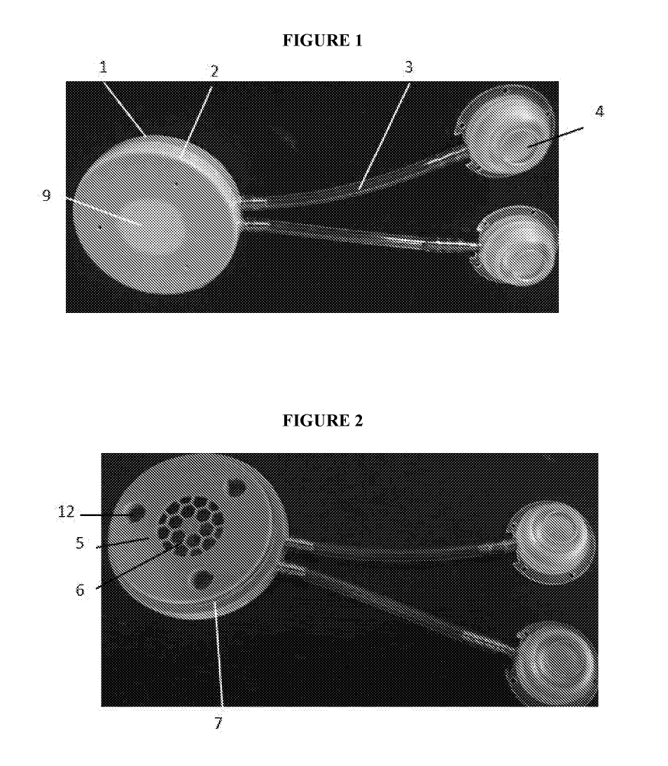

The composite membrane was produced by gluing a two layers of hydrophilic porous membrane (9), (e.g. Biopore) to the cover (2) by use of medical Epoxy (e.g., but not limited to, Epotek 301-2) (FIG. 1). In some embodiments, the hydrophilic porous membrane is made from PTFE. In some embodiments, the hydrophilic porous membrane has varying pore sizes. In some embodiments, the hydrophilic porous membrane has pores not exceeding 0.4 μm in size. In some embodiments, the size of pores in the hydrophilic porous membrane is between 0.1 μm and 0.4 μm. In some embodiments, the volume of the hydrophilic porous membrane is about 90% voids. In some embodiments, the volume of the hydrophilic porous membrane is between 80% and 95% voids. As used herein, the term “hydrophilic” is used to denote a membrane that readily absorbs water. In some embodiments, a hydrophilic porous membrane is a Biopore membrane. In some embodiments, a hydrophilic porous membrane is a membrane which, over the course of its useful life, does not require a wetting step before use. In some embodiments, a hydrophilic porous membrane is a membrane which suffers little or no loss of membrane material due to extraction over multiple wet-dry cycles. In some embodiments, a hydrophilic porous membrane is a membrane which has a critical wetting surface tension of at least 72 dynes per centimeter.

In some embodiments, one layer is used. In some embodiments, three layers are used. In some embodiments, four layers are used. In some embodiments, five layers are used. In some embodiments, each layer of the hydrophilic porous membrane is 20 μm in thickness. In some embodiments, each layer of the hydrophilic porous membrane is 40 μm in thickness. In some embodiments, each layer of the hydrophilic porous membrane is between 20 μm and 100 μm in thickness. In some embodiments, the total thickness of the layers of membrane that are used is between 20 μm and 100 μm. In some embodiments, the total thickness is 20 μm. In some embodiments, the total thickness is 40 μm. In some embodiments, the total thickness is 60 μm. In some embodiments, the total thickness is 80 μm. In some embodiments, the total thickness is 100 μm. In some embodiments, the total thickness is between 20 μm and 100 μm. In some embodiments, the total thickness is between 40 μm and 100 μm. In some embodiments, the total thickness is between 60 μm and 100 μm. In some embodiments, the total thickness is between 80 μm and 100 μm. In some embodiments, the total thickness is between 20 μm and 80 μm. In some embodiments, the total thickness is between 40 μm and 80 μm. In some embodiments, the total thickness is between 60 μm and 80 μm. In some embodiments, the total thickness is between 20 μm and 60 μm. In some embodiments, the total thickness is between 40 μm and 60 μm. In some embodiments, the total thickness is between 20 μm and 40 μm.

HM alginate was impregnated into the hydrophilic porous double membrane (e.g., but not limited to two or more membranes, Biopore™, a PTFE membrane, or equivalent having a pore size of 0.4 μm each and a membrane width of between 25 and 75 μm) by dissolving 6% HM alginate in a HTK solution (Histidine-tryptophan-ketoglutarate, or Custodiol HTK solution is a high-flow, low-potassium preservation solution used for organ transplantation) and applying pressure (i.e., impregnating) the HM alginate into the membrane pores by use of a vacuum at 0.03 mbar, where the vacuum was applied for 2 to 10 minutes. The membranes were then incubated for an additional 10 minutes without a vacuum, and any remaining unimpregnated alginate was wiped from the membranes. In some embodiments, the amount of alginate solution is sufficient to saturate the membrane. In some embodiments, the amount of alginate solution is at least 0.9 times the volume of the membrane.

In another exemplary embodiment, HM alginate of second-outer alginate structure was layered on either side of membrane. The alginate hydrogel of second, outer alginate structure was cross-linked by immersing the membrane-alginate system in 20-60 mM Barium chloride solution (e.g., typically 30 mM) for a period of 5-60 min (e.g., typically 12-16 min). As a result, the second, outer alginate layer included a membrane-alginate layer, where the membrane comprises a physical porous membrane in which the pores of membrane were impregnated with cross-linked HM alginate.

The HM alginate was cross-linked by immersing the cover in a barium solution for a period of, but not limited to, 22 min (e.g., between 15-30 minutes).

The wet cover was freeze-dried by lyophilization, so as to result in maintaining the structure of the alginate and to increase the shelf-life of the composite membrane (e.g., but not limited to between 1 month-120 months) and to allow fast rehydration of the membrane upon integration of the tissue

As illustrated in FIGS. 3 and 7, to integrate the cells and/or tissue (e.g. islets of Langerhans), the cells and/or tissue are mixed with 3.5% HG alginate, located in the grove (10) inside of the cover (2) and cross-linked with strontium for 22 min. In some embodiments, the cross-linking can be performed in e.g., but not limited to, 1-60 minutes. In some embodiments, the cross-linking can be performed in e.g., but not limited to, 5-35 minutes. In some embodiments, the cross-linking can be performed in e.g., but not limited to, 5-25 minutes. In some embodiments, the cross-linking can be performed in e.g., but not limited to, 5-15 minutes. In some embodiments, the cross-linking can be performed in e.g., but not limited to, 15-35 minutes. In some embodiments, the cross-linking can be performed in e.g., but not limited to, 25-35 minutes.

As shown in FIG. 4, the cover is closed on the body of the apparatus by inserting Huber needles into the holes (7) through the silicon rubber plugs (8). The cover (2) is closed on the body (1) and the air locked between the cover (2) and the body (1) escaped through the needles. The needles are removed after closing the apparatus. Additionally, the air locked between the cover and the body can be eliminated by vacuum. In an air tight chamber, the cover is placed on the body, a milled vacuum of 1.1 atm to 2.0 atm (where 1.0 atm is the ambient pressure) is applied and the cover is closed on the body of the device.

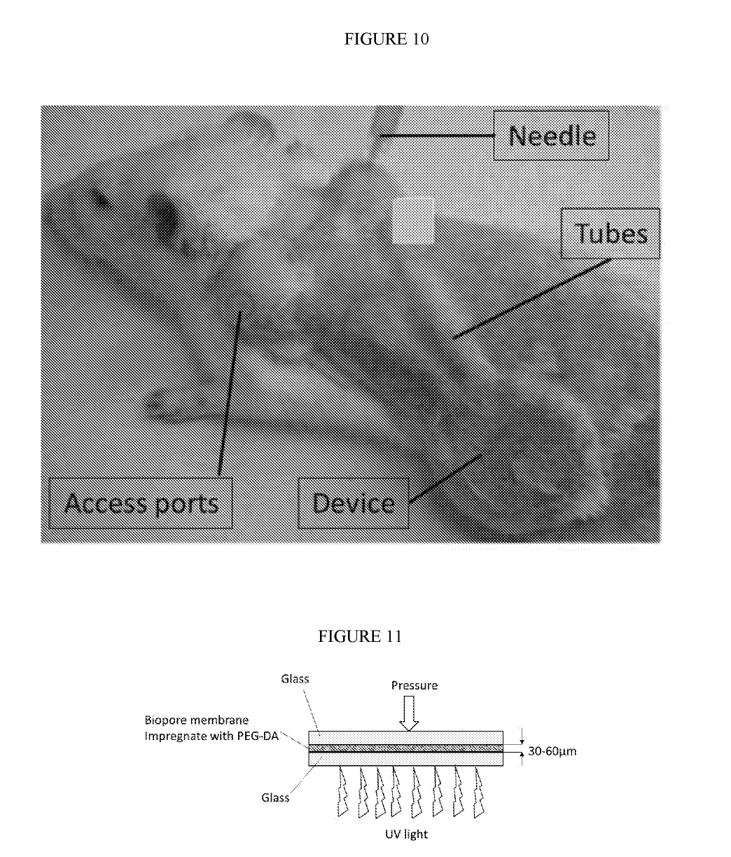

The gas mixture (i.e., oxygen mixture) is replenished every 24 hours by inserting two 24 G Huber needles into the ports (as shown as (4) in FIG. 1 and “Needle” in FIG. 10) and 15 ml of the gas mixture injected into the device at a rate of 15 ml/min.

In some embodiments, the apparatus of the present invention includes HG alginate, where the HG alginate allows for immobilization of islets cells/tissue, such that the graft does not penetrate into the hydrophilic PTFE porous membrane.

FIG. 1 illustrates an embodiment of the apparatus of the present invention. (1) Body of the device containing the gas chamber. (2) Cover of the device containing the composite dry membrane (9). (3) Tube connecting the gas chamber with the ports (4).

FIG. 2 illustrates an embodiment of the apparatus of the present invention, shown without the cover. (5) Body of the device containing the gas chamber. (6) Gas permeable membrane. (7) O-Ring sealing. (12) Holes for the cover.

FIGS. 3A and 3B illustrate an embodiment of the apparatus of the present invention, showing the cover of the apparatus. FIG. 3A: (7) holes for needle. (9) Composite membrane FIG. 3B: (8) silicon rubber plugs. (9) Composite membrane under the grove for the cells or tissue.

FIG. 4 illustrates an embodiment of the apparatus of the present invention, showing the closure of the apparatus. Cover (2) loaded with gel mix with tissue or cells (10) are closed on the body (1). O-ring (7) prevents body liquid to penetrate the device.

### Example 2: Process Description of Integrating a Membrane

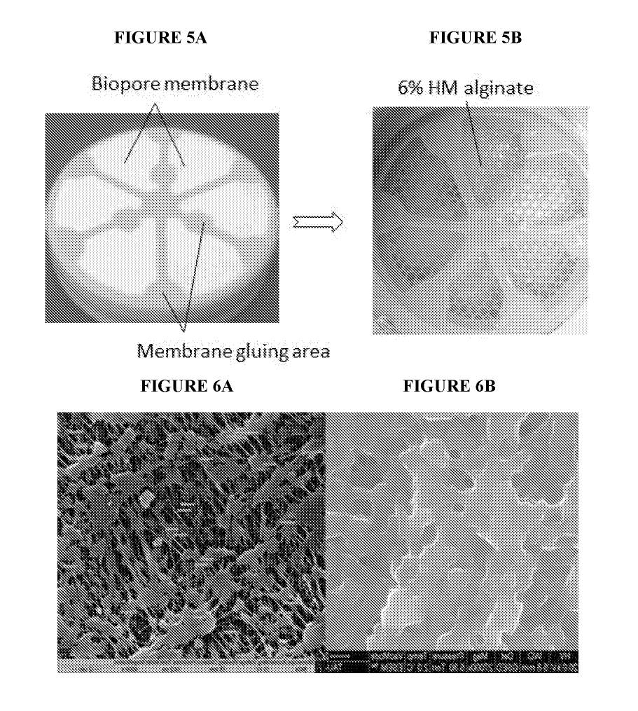

In some embodiments, the apparatus of the present invention was manufactured by first performing an integration step, where 4-8% HM alginate dissolved in HTK was placed on top of a hydrophilic PTFE porous membrane (Biopore), as shown in FIG. 5A. FIG. 5A shows a top view of the apparatus with hydrophilic PTFE (Biopore) porous membrane glued on a plastic frame of the implanted device. FIG. 5B shows 4-8% HM alginate placed on the Biopore membrane. Then, to impregnate the membrane, the 4-8% HM alginate was forced into the PTFE porous membrane by a low vacuum pressure (e.g., but not limited to, between 0.025 and 0.25 mPa). Extra alginate was wiped from the surface of the membrane. The 4-8% HM alginate, now part of the complex PTFE membrane, was cross-linked with barium chloride solution. The PTFE membrane containing the HM alginate was lyophilized to achieve dry and stable characteristics. The composite membrane contains the following pores: (a) micro-pores formed in the manufacturing process of the hydrophilic PTFE membrane and (b) nano-pores created by the HM-alginate polymers. The resulting membrane generated by the process is a dry product, where the pores of the PTFE membrane are completely filled with HM alginate, as shown in FIGS. 6A and 6B. FIGS. 6A and 6B show an exemplary embodiment of the present invention, showing an ESEM of membrane surfaces, where FIG. 6A shows the PTFE (Biopore) membrane as is and FIG. 6B shows the Biopore membrane impregnated with HM alginate. The dry composite was sterilized by Ethylene Oxide at 32° C. for 18 h and can be stored for prolonged periods of time, e.g., but not limited to, 1 month, 3 months, 6 months, 9 months, 12 months, 18 months, 24 months, etc.

In some embodiments, to integrate, the graft containing islet cells (i.e., functional cells) was mixed with HG-alginate, loaded onto the PTFE membrane and the hydrogel was cross-linked by use of Strontium Chloride solution, e.g., (70 mM) for 22 minutes. During this process, as shown in FIG. 7, the extraneous composite PTFE membrane was hydrated. As the pores in the PTFE membrane (Biopore) were previously filled with cross-linked HM alginate, the HG alginate and/or the tissue mixed did not penetrate into the cross-linked HM alginate. Accordingly, this process resulted in two separate compositions, as shown in FIG. 7 as a schematic overview of the integration process (drawing not to scale): (i) Strontium cross-linked HG alginate containing the islets graft and (ii) Barium cross-linked HM alginate reinforced by hydrophilic PTFE membrane.

In some embodiments, the two alginate layers (i.e., the HM alginate and the HG alginate) are two separate components that can be taken apart from the apparatus and remain substantially intact when separating from the apparatus.

### Example 3: Process Description of Impregnating a Membrane

In some embodiments, an exemplary apparatus is formed by a process including impregnating a polymer into a hydrophilic porous membrane. In some embodiments, the membrane has properties as described above with reference to Example 1. In some embodiments, the polymer includes a polyether. In some embodiments, the polymer includes PEG. In some embodiments, the polymer includes PEG-DA. In some embodiments, the polymer includes PEG-A. In some embodiments, the polymer includes PEG-DMA. The term “polyether” will be used herein to refer collectively to the class of polymers including, but not limited to, PEG, PEG-DA, PEG-A, and PEG-DMA. In some embodiments, the polymer includes a 4-arm polyether. In some embodiments, the polymer includes an 8-arm polyether. In some embodiments, the polymer includes a 16-arm polyether.

In some embodiments, a 10% solution of PEG-DA 10 kDa (dissolved in phosphate-buffered saline (“PBS”) with 0.1% of a photoinitiator (i.e., a polyether solution) was spread onto a Biopore membrane. In some embodiments, the amount of the polyether solution is sufficient to saturate the membrane. In some embodiments, the amount of the polyether solution is at least 0.9 times the volume of the membrane. In some embodiments, the photoinitiator is 2-Hydroxy-1-[4-(2-hydroxyethoxy) phenyl]-2-methyl-1-propanone. In some embodiments, the photoinitiator is the photoinitiator distributed by BASF under the trade name IRGACURE 2959. In some embodiments, the photoinitiator is another suitable photoinitiator that is sensitive to UV light. In some embodiments, the photoinitiator is another suitable photoinitiator that is biocompatible and sensitive to UV light. In some embodiments, the membrane was then spread onto transparent glass (i.e., a transparent material), covered with additional transparent glass, and compressed between the two glasses under an applied pressure of 0.025 MPa for five (5) minutes. In some embodiments, a pressure greater than 0.025 MPa is applied. In some embodiments, the pressure is sufficient to cause the polyether solution to saturate the membrane. In some embodiments, during compression, the membrane is cured with ultraviolet (UV) light. In some embodiments, the level of transparency of the glass is sufficient such that an applied UV light causes curing of the polyether solution. FIG. 11 shows the membrane compressed between the two glasses during the curing process. In some embodiments, during compression, the membrane was cured with UV light illuminated at a wavelength of 365 nm and an intensity of 850 W/cm². In some embodiments, during compression, the membrane was cured with UV light illuminated at a wavelength of 405 nm and an intensity of 950 W/cm². In some embodiments, the polyether solution is impregnated into the membrane by application of a vacuum, as described above.

In some embodiments, the compression and curing results in a composite membrane having a thickness of between 30 and 60 microns. In some embodiments, the composite membrane is dried in an oven at 70° C. In some embodiments, the composite membrane is dried by lyophilization in accordance with the following table:

| | | | | Temper- | | Safety |
|---|---|---|---|---|---|---|
| | | | Min- | ature | Pressure | Pressure |
| Step | Process-Phase | Hours | utes | (° C.) | (mBar) | (mBar) |
| 1 | Start values | 0 | 0 | 10 | OFF | OFF |
| 2 | Pre-Freezing | 0 | 10 | 5 | OFF | OFF |
| 3 | Pre-Freezing | 0 | 20 | 5 | OFF | OFF |
| 4 | Freezing | 0 | 10 | 0 | OFF | OFF |
| 5 | Freezing | 0 | 35 | 0 | OFF | OFF |
| 6 | Freezing | 0 | 10 | (−5) | OFF | OFF |
| 7 | Freezing | 0 | 35 | (−5) | OFF | OFF |
| 8 | Freezing | 0 | 10 | (−10) | OFF | OFF |
| 9 | Freezing | 0 | 35 | (−10) | OFF | OFF |
| 10 | Freezing | 0 | 10 | (−15) | OFF | OFF |
| 11 | Freezing | 0 | 35 | (−15) | OFF | OFF |
| 12 | Freezing | 0 | 10 | (−20) | OFF | OFF |
| 13 | Preparation | 0 | 35 | (−20) | OFF | OFF |
| 14 | Sublimation | 0 | 01 | (−20) | 0.250 | 0.520 |
| 15 | Sublimation | 15 | 59 | (−20) | 0.250 | 0.520 |
| 16 | Secondary drying | 0 | 10 | (−10) | 0.080 | 0.520 |
| 17 | Secondary drying | 0 | 50 | (−10) | 0.080 | 0.520 |
| 18 | Secondary drying | 0 | 10 | 0 | 0.080 | 0.520 |
| 19 | Secondary drying | 0 | 50 | 0 | 0.080 | 0.520 |
| 20 | Secondary drying | 0 | 10 | 10 | 0.080 | 0.520 |
| 21 | Secondary drying | 0 | 50 | 10 | 0.080 | 0.520 |
| 22 | Secondary drying | 0 | 10 | 20 | 0.080 | 0.520 |
| 23 | Secondary drying | 1 | 50 | 20 | 0.080 | 0.520 |

In some embodiments, the polyether has a mass of about 10 kDa. In some embodiments, the polyether has a mass of about 3.35 kDa. In some embodiments, the polyether has a mass of about 6.0 kDa. In some embodiments, the polyether has a mass of about 8.0 kDa. In some embodiments, the polyether has a mass of about 20 kDa. In some embodiments, the polyether has a mass of between 3.35 kDa and 20 kDa. In some embodiments, the polyether has a mass of between 3.35 kDa and 10 kDa. In some embodiments, the polyether has a mass of between 3.35 kDa and 8.0 kDa. In some embodiments, the polyether has a mass of between 3.35 kDa and 6.0 kDa. In some embodiments, the polyether has a mass of between 6.0 kDa and 20 kDa. In some embodiments, the polyether has a mass of between 6.0 kDa and 10 kDa. In some embodiments, the polyether has a mass of between 6.0 kDa and 8.0 kDa. In some embodiments, the polyether has a mass of between 8.0 kDa and 20 kDa. In some embodiments, the polyether has a mass of between 8.0 kDa and 10 kDa. In some embodiments, the polyether has a mass of between 10 kDa and 20 kDa.

In some embodiments, the polyether solution has a concentration of about 5%. In some embodiments, the polyether solution has a concentration of about 10%. In some embodiments, the polyether solution has a concentration of about 15%. In some embodiments, the polyether solution has a concentration of about 20%. In some embodiments, the polyether solution has a concentration of between 5% and 20%. In some embodiments, the polyether solution has a concentration of between 5% and 15%. In some embodiments, the polyether solution has a concentration of between 5% and 10%. In some embodiments, the polyether solution has a concentration of between 10% and 20%. In some embodiments, the polyether solution has a concentration of between 10% and 15%. In some embodiments, the polyether solution has a concentration of between 15% and 20%.

In some embodiments, the photoinitiator has a concentration of between 0.05% and 0.3%. In some embodiments, the photoinitiator has a concentration of between 0.05% and 0.25%. In some embodiments, the photoinitiator has a concentration of between 0.05% and 0.2%. In some embodiments, the photoinitiator has a concentration of between 0.05% and 0.15%. In some embodiments, the photoinitiator has a concentration of between 0.05% and 0.1%. In some embodiments, the photoinitiator has a concentration of between 0.1% and 0.3%. In some embodiments, the photoinitiator has a concentration of between 0.1% and 0.25%. In some embodiments, the photoinitiator has a concentration of between 0.1% and 0.2%. In some embodiments, the photoinitiator has a concentration of between 0.1% and 0.15%. In some embodiments, the photoinitiator has a concentration of between 0.15% and 0.3%. In some embodiments, the photoinitiator has a concentration of between 0.15% and 0.25%. In some embodiments, the photoinitiator has a concentration of between 0.15% and 0.2%. In some embodiments, the photoinitiator has a concentration of between 0.2% and 0.3%. In some embodiments, the photoinitiator has a concentration of between 0.2% and 0.25%. In some embodiments, the photoinitiator has a concentration of between 0.25% and 0.3%.

In some embodiments, the volume ratio of hydrogel to membrane in the composite membrane is about 9 to 1. In some embodiments, the volume ratio of hydrogel to membrane in the composite membrane is between 4 to 1 and 20 to 1. In some embodiments, the volume ratio of hydrogel to membrane in the composite membrane is between 4 to 1 and 9 to 1. In some embodiments, the volume ratio of hydrogel to membrane in the composite membrane is between 9 to 1 and 20 to 1. In some embodiments, the total thickness of the composite membrane is between 20 μm and 100 μm. In some embodiments, the total thickness is 25 μm. In some embodiments, the total thickness is 50 μm. In some embodiments, the total thickness is 75 μm. In some embodiments, the total thickness is 100 μm. In some embodiments, the total thickness is between 25 μm and 100 μm. In some embodiments, the total thickness is between 50 μm and 100 μm. In some embodiments, the total thickness is between 75 μm and 100 μm. In some embodiments, the total thickness is between 50 μm and 100 μm. In some embodiments, the total thickness is between 50 μm and 75 μm. In some embodiments, the total thickness is between 75 μm and 100 μm. In some embodiments, the water content of the hydrogel is between 80% and 99%.

In some embodiments, a composite membrane that has been prepared as described above may be suitable for use as an immune barrier. In some embodiments, the composite membrane allows nutrients and products, such as insulin and glucagon, to pass therethrough. In some embodiments, the composite membrane prevents immune cells and immune proteins, such as immunoglobulin-G and C1q, from passing therethrough. In some embodiments, the composite membrane is opaque when dehydrated (e.g., after oven-drying or lyophilization). In some embodiments, the composite membrane is at least partially transparent when rehydrated.

In some embodiments, to integrate, a graft containing functional cells (e.g., islet cells) was mixed with a polymer, loaded onto the composite membrane, and the hydrogel was cross-linked by use of a cross-linking solution. In some embodiments, the cross-linking solution is a 70 mM strontium chloride solution. In some embodiments, cross-linking is performed for 22 minutes. In some embodiments, cross-linking is performed for between 20 and 30 minutes. In some embodiments, the scaffold of the composite PTFE porous membrane is hydrated. As the pores in the composite were previously filled with a cross-linked hydrogel, mixture of polymer and functional cells does not penetrate into the composite membrane. Accordingly, this process results in two separate compositions: the composite membrane and a separate cross-linked hydrogel containing functional cells.

### Example 4: Process Description of Cell Integration

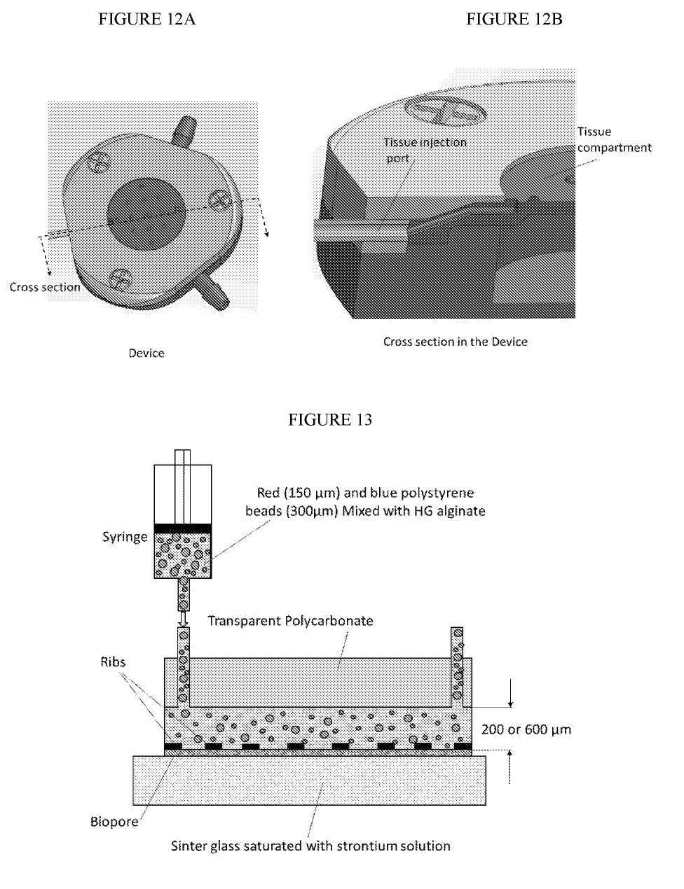

In some embodiments, an exemplary apparatus is formed by a process including a cell integration step. In some embodiments, functional cells (e.g., islets of Langerhans, human stem cells, adrenal cells, etc.) are mixed with a non-crosslinked or non-cured hydrogel (e.g., HG alginate, PEG-DA, etc.). In some embodiments, the mixture so produced is injected into an implantable device by injection into a specialized tissue injection port leading to a tissue compartment. FIG. 12A shows an exemplary implantable device, while FIG. 12B shows a cross-sectional view of the implantable device of FIG. 12A, taken along the cross-section indicated in FIG. 12A, and showing the locations of the tissue injection port and the tissue compartment. In some embodiments, after the mixture has been injected, the hydrogel is cross-linked. In some embodiments, cross-linking is performed by immersing the device in a cross-linking solution. In some embodiments, a cross-linking solution includes strontium. In some embodiments, a cross-linking solution includes barium. In some embodiments, a cross-linking solution includes calcium. In some embodiments, the hydrogel is cross-linked by curing through illumination with UV light.

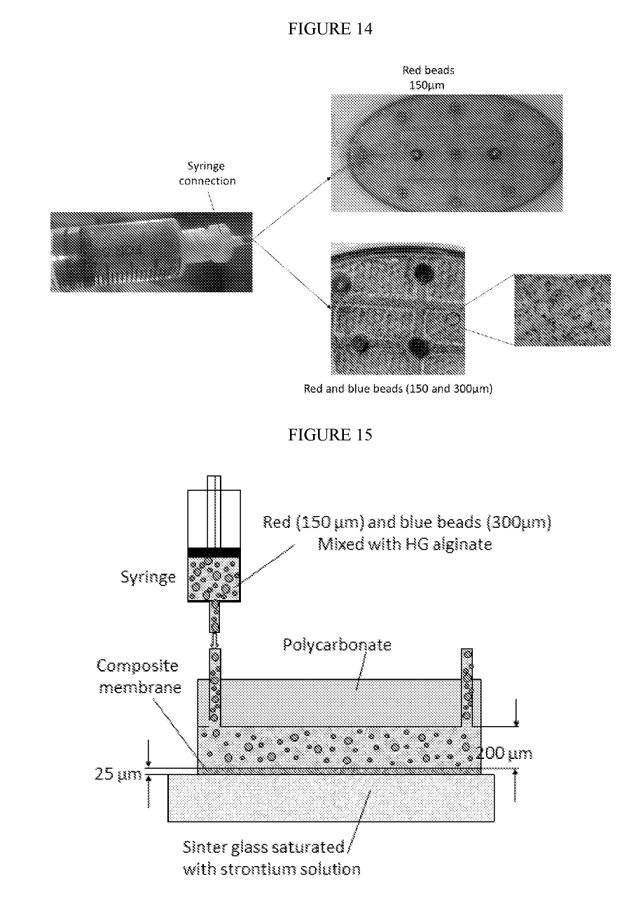

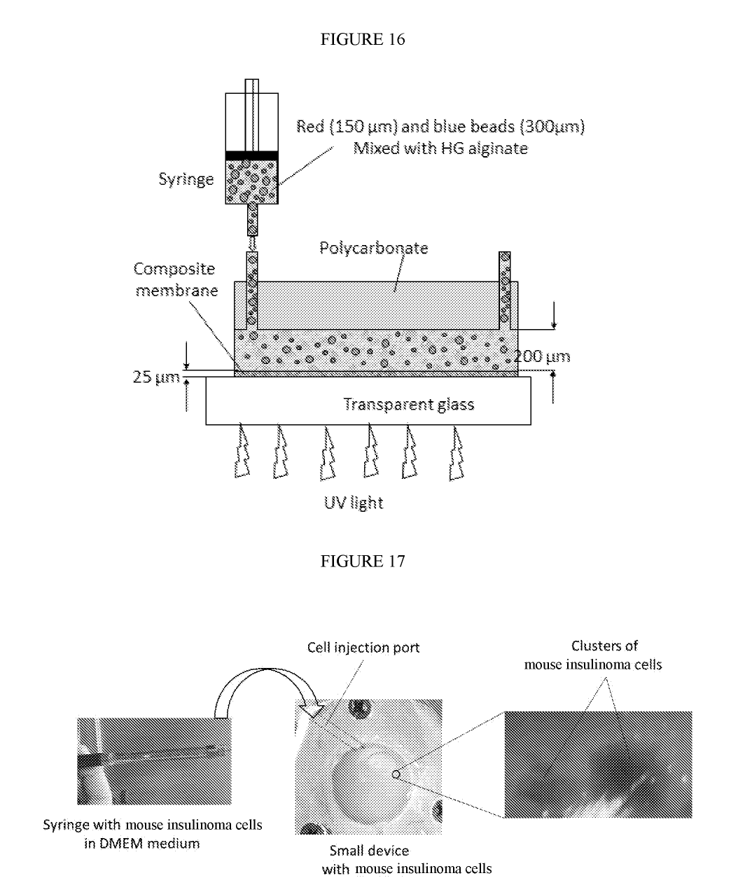

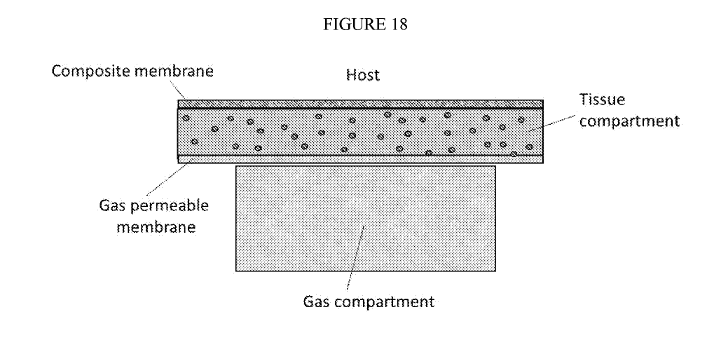

In some embodiments, an exemplary apparatus is formed by a process including a cell integration step not involving the use of a hydrogel. In some embodiments, functional cells (e.g., islets of Langerhans, human stem cells, adrenal cells, etc.) are cultured in a basal medium. In some embodiments, the basal medium is Dulbecco's Modified Eagle Medium (DMEM). In some embodiments, functional cells so cultured are placed into a syringe and injected into a tissue chamber (alternately referred to as a tissue compartment) of an implantable device (see FIGS. 13 and 14). In some embodiments (See FIG. 16), the tissue chamber is positioned such that, when functional cells are injected into the tissue chamber, a composite membrane is attached to the implantable device, and the implantable device is implanted into a host, the composite membrane is positioned between the tissue compartment and tissue of the host.

### Experimental Results

### Removal of Apparatus

In one experiment, a claimed implantable device was implanted in a pig for ninety (90) days. Before implantation, the alginate content of the tissue-alginate layer was 5,346 μg/mm3. After retrieval, the alginate content of the tissue-alginate layer was 5,300 μg/mm3. This demonstrated that the alginate content of the tissue-alginate layer remained substantially intact.

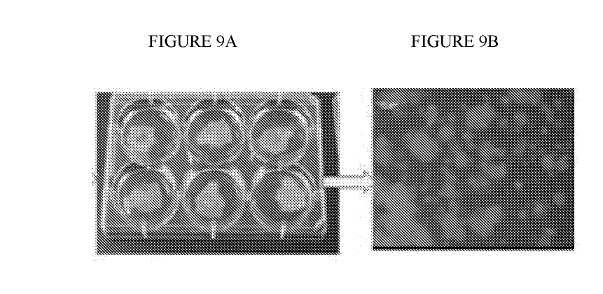

In another experiment, a claimed implantable device was implanted in a patient for ten (10) months. After removal, experiments revealed that the quantity of alginate of the membrane-alginate structure was similar before implantation and after removal of the device (22.8±1.4 and 22.1±1.3, respectively). No cells were noticed in the membrane-alginate structure upon retrieval of the device, demonstrating that the cells did not migrate into the membrane-alginate structure. The photographs below show the inside of an implantable device with the membrane-alginate structure (FIGS. 8A and 8B (a close-up of FIG. 8A) and that no cells were found in the membrane-alginate structure. Further, the tissue-alginate layer comprising cells was totally retrievable from the device, demonstrating that the membrane-alginate structure and the tissue-alginate layer are two separate components. FIGS. 9A and 9B show that after a ten (10) month implantation, the tissue-alginate layer comprising the cells can be completely retrieved (left picture, tissue-alginate layers in the six-well plate) and the cells are located only in the tissue-alginate layers.

### Replenishing Gas Mixture in Rats

Rats having the implanted apparatus were replenished with a gas mixture. Every 24 hours, each rat was sedated, two 27 G Huber needles were inserted into each of the two implanted access ports, as shown in FIG. 10 (although only one needle is shown in this picture), and the tank was purged with 10-15 ml (about 3 to 5 tank volumes) of gas mixture containing the specified oxygen concentrations, 40 mmHg CO2, and balance N2. The final total pressure in the tank was equal to ambient atmospheric pressure. To obtain the different oxygen mixtures, prefilled cylinders were used (Maxima, Israel). FIG. 10 shows the position of the implanted system in reference to the tubes, apparatus/device, and access ports for gas replenishment. After replenishing the oxygen (i.e., having a measurement of 1 atm), the needle is removed and the gas is pushed into the device until a predetermined internal pressure is reached. In a similar example, in a device configured for use in humans, 1.4 atms in the device is achieved by injecting 9 ml of gas.

### Immune Barrier

In one experiment, a composite membrane prepared as described above with reference to FIG. 11 was placed in a diffusion cell. Diffusion rates of glucose, human insulin, and rat immunoglobulin-G (IgG) through the composite membrane were measured. Diffusion rates of glucose, human insulin, and rat IgG through an empty membrane (i.e., a Biopore membrane not treated as described above with reference to FIG. 11) were considered as a reference. The diffusion rates of glucose and human insulin through the composite membrane were similar to those through the empty membrane. In some embodiments, the diffusion constant of glucose through the composite membrane is between 1.3*10⁻⁶ cm²/sec and 1.5*10⁻⁶ cm²/sec. In some embodiments, the diffusion constant of insulin through the composite membrane is between 1.5*10⁻⁷ cm²/sec and 1.7*10⁻⁷ cm²/sec. The diffusion rate of rat IgG through the composite membrane was almost completely blocked as compared to the rate through the empty membrane. In some embodiments, the transfer rate of rat IgG through the composite membrane is between 0.05% and 0.1% transferred from the source cell to the sink cell for 48 hours. Consequently, it may be seen that a Biopore membrane impregnated with 10 kDa PEG-DA can be used as an immune barrier. In some embodiments, a device includes a hydrophilic PTFE porous membrane impregnated with PEG-DA as an immune barrier. In some embodiments, small molecules (i.e., molecules having a molecular weight less than about 900 Da, such as glucose) pass freely through the composite membrane. In some embodiments, medium molecules (i.e., molecules having a molecular weight between about 900 Da and about 100,000 Da, such as insulin) pass through the composite membrane with a slight delay, the delay varying based on the molecular weight of the molecules. In some embodiments, substantially all large molecules (i.e., molecules having a molecular weight of greater than about 100,000 Da, such as IgG) are prevented from passing through the composite membrane.

In some embodiments, the PEG-DA has a mass of 10 kDa. In some embodiments, the PEG-DA has a mass of about 10 kDa. In some embodiments, the PEG-DA has a mass of between 3.35 kDa and 20 kDa. In some embodiments, the PEG-DA has a mass of between 3.35 kDa and 10 kDa. In some embodiments, the PEG-DA has a mass of between 3.35 kDa and 8.0 kDa. In some embodiments, the PEG-DA has a mass of between 3.35 kDa and 6.0 kDa. In some embodiments, the PEG-DA has a mass of between 6.0 kDa and 20 kDa. In some embodiments, the PEG-DA has a mass of between 6.0 kDa and 10 kDa. In some embodiments, the PEG-DA has a mass of between 6.0 kDa and 8.0 kDa. In some embodiments, the PEG-DA has a mass of between 8.0 kDa and 20 kDa. In some embodiments, the PEG-DA has a mass of between 8.0 kDa and 10 kDa. In some embodiments, the PEG-DA has a mass of between 10 kDa and 20 kDa.

### Injection of Cells

In one experiment, an injection system was tested as illustrated in FIGS. 13 and 14. FIG. 13 shows an illustration of an injection system, while FIG. 14 shows various images of the experimental injection system. A flat ellipsoid device having a major axis length of 11.5 cm and a minor axis length of 7.0 cm was equipped with two loading ports in one side and two discharge ports in the other side (see FIG. 14, top right panel). A 600 μm thickness tissue chamber was created by gluing two layers of a porous membrane (0.4 μm pore diameter Biopore) with a thickness of 50 μm with an epoxy adhesive onto ribs made of polyether ether ketone (PEEK) to one side of the tissue chamber, with transparent polycarbonate forming the other side of the tissue chamber. The porous membrane was placed on flat sinter glass at the bottom of the system so as to maintain a constant thickness. Polystyrene beads were immersed in polysorbate 20 solution for 48 hours, washed, and mixed with 3.5% HG alginate dissolved in HTK solution. In one experiment, all the polystyrene beads had a diameter of 150 μm. In another experiment, the polystyrene beads were a mixture of 150 μm and 300 μm beads. The mixture of beads and alginate was loaded into a 10 mL syringe (see FIG. 14, left panel) and injected into the tissue chamber at a rate of 3.5 mL per minute. The sinter glass was immersed in 70 mM strontium solution for 25 minutes and washed with a 4-(2-hydroxyethyl)-1-piperazineethanesulfonic acid solution to perform cross-linking of the HG alginate. The injected mixture including only 150 μm beads is shown in FIG. 14, top panel. The injected mixture including 150 μm and 300 μm beads is shown in the bottom center panel, and in a magnified view in the bottom right panel. In both cases, the mixture was uniformly injected into the chamber without air bubbles.

Referring now to FIG. 15, in a second experiment, a similar tissue chamber to that described above, but having a thickness of 200 μm rather than 600 μm, was created by gluing two layers of a porous membrane (0.4 μm pore diameter Biopore) with a thickness of 50 μm with an epoxy adhesive onto flat sinter glass to one side of the tissue chamber so as to maintain a constant thickness, with transparent polycarbonate forming the other side of the tissue chamber. Mixed 150 μm and 300 μm polystyrene beads were immersed in polysorbate 20 solution for 48 hours, washed, and mixed with 3.5% HG alginate dissolved in HTK solution. The mixture of beads and alginate was loaded into a 10 mL syringe and injected into the tissue chamber at a rate of 3.5 mL per minute. The sinter glass was immersed in 70 mM strontium solution for 25 minutes and washed with a 4-(2-hydroxyethyl)-1-piperazineethanesulfonic acid solution to perform cross-linking of the HG alginate. The mixture was uniformly injected into the chamber without air bubbles and the HG alginate was cross-linked by the strontium solution.

Referring now to FIG. 16, in a third experiment, a similar tissue chamber to that described above with reference to FIG. 15, but using transparent glass rather than sinter glass, was created. Two layers of a porous membrane (0.4 μm pore diameter Biopore) with a thickness of 50 μm were glued to transparent glass with an epoxy adhesive to form side of the tissue chamber so as to maintain a constant thickness, with transparent polycarbonate forming the other side of the tissue chamber. Mixed 150 μm and 300 μm polystyrene beads were immersed in polysorbate 20 solution for 48 hours, washed, and mixed with a polyether solution (e.g., 10% PEG-DA 10 kDa dissolved in phosphate-buffered saline (“PBS”) with 0.1% of a photoinitiator). The mixture of beads and polyether was loaded into a 10 mL syringe and injected into the tissue chamber at a rate of 3.5 mL per minute. UV light was cast on the transparent glass to cure the polyether. The mixture was uniformly injected into the chamber without air bubbles.

In a fourth experiment, the tissue chamber described above with reference to FIG. 15 was created. 20×10⁶ mouse insulinoma cells were cultured in Dulbecco's Modified Eagle Medium (“DMEM”) and supplements. The cultured cells were loaded into a syringe and injected into the tissue chamber. The mixture was uniformly injected into the chamber without air bubbles. FIG. 17 shows various views of the injection process described above with reference to FIG. 15, as performed using the cultured mouse insulinoma cells.

Publications cited throughout this document are hereby incorporated by reference in their entirety. Although the various aspects of the invention have been illustrated above by reference to examples and preferred embodiments, it will be appreciated that the scope of the invention is defined not by the foregoing description but by the following claims properly construed under principles of patent law.

What is claimed is:

1. A method, comprising:
spreading a solution on a hydrophilic porous membrane, the solution including a polyether and a photoinitiator;
impregnating the hydrophilic porous membrane with the solution; and
curing the solution located within the hydrophilic porous membrane by exposure to ultraviolet light to produce a composite membrane.

2. The method of claim 1, wherein the composite membrane has a suitable pore size such that molecules having a molecular weight of greater than about 100,000 Daltons are prevented from passing through the membrane.

3. The method of claim 1, further comprising:
drying the composite membrane by either oven-drying or lyophilizing.

4. The method of claim 3, further comprising:
placing the dried composite membrane in an implantable device.

5. The method of claim 4, further comprising:
prior to the step of placing the dried composite membrane in the implantable device, performing the steps of:
mixing functional cells with a polymer to produce a cell mixture;
placing the cell mixture on the composite membrane; and
cross-linking the cell mixture with a cross-linking agent to produce an embedded cell layer adjacent the composite membrane,
wherein, when the dried composite membrane is placed in the implantable device, the embedded cell layer is also placed in the implantable device.

6. The method of claim 5, wherein the cross-linking agent includes at least one of barium, strontium, and calcium.

7. The method of claim 5, wherein the functional cells include at least one of islets of Langerhans, stem cells, and adrenal cells.

8. The method of claim 5, wherein the implantable device is configured to receive a supply of oxygen from an external source.

9. The method of claim 8, wherein the composite membrane and the embedded cell layer are positioned in the implantable device such that the composite membrane is positioned between the external oxygen source and the embedded cell layer.

10. The method of claim 4, further comprising:
culturing functional cells in a basal medium; and
injecting the cultured functional cells and the basal medium into a tissue chamber of the implantable device.

11. The method of claim 1, wherein the functional cells include at least one of islets of Langerhans, stem cells, and adrenal cells.

12. The method of claim 10, wherein the implantable device is configured to receive a supply of oxygen from an external source.

13. The method of claim 12, wherein the composite membrane and the tissue chamber are positioned in the implantable device such that, when the implantable device is implanted within a host, the composite membrane is positioned between the tissue chamber and tissue of the host.

14. The method of claim 1, wherein the polyether includes at least one of polyethylene glycol diacrylate, polyethylene glycol acrylate, and polyethylene glycol dimethacrylate.

15. The method of claim 1, wherein the step of impregnating the hydrophilic porous membrane with the solution includes compressing the hydrophilic porous membrane and the solution between two pieces of a transparent material to impregnate the hydrophilic porous membrane with the solution, and wherein the curing step is performed while the hydrophilic porous membrane and the solution are compressed between the two pieces of the transparent material.

16. A method, comprising:
placing an HM alginate solution on a hydrophilic porous membrane;
exposing the HM alginate solution on the hydrophilic porous membrane to a vacuum pressure to produce a hydrophilic porous membrane impregnated with HM alginate;
exposing the hydrophilic porous membrane impregnated with HM alginate to a cross-linking solution to cross-link the HM alginate and produce a hydrophilic porous membrane impregnated with cross-linked HM alginate; and
lyophilizing the hydrophilic porous membrane impregnated with cross-linked HM alginate to produce a composite membrane.

17. The method of claim 16, wherein the cross-linking solution includes at least one of strontium, barium, and calcium.

18. The method of claim 16, further comprising:
mixing functional cells with HG-alginate to produce a cell mixture;
placing the cell mixture on the composite membrane; and
cross-linking the cell mixture with a cross-linking agent to produce an embedded cell layer adjacent to the composite membrane.

19. The method of claim 18, wherein the cross-linking agent includes at least one of strontium, barium, and calcium.

20. The method of claim 18, further comprising:
installing the composite membrane and the embedded cell layer in an implantable device that is configured to receive oxygen from an external oxygen source, wherein the composite membrane and the embedded cell layer are positioned in the implantable device such that the composite membrane is positioned between the external oxygen source and the embedded cell layer.

21. The method of claim 18, wherein the functional cells include at least one of islets of Langerhans, stem cells, and adrenal cells.

22. The method of claim 18, further comprising:
immobilizing biologically active molecules in the HG-alginate.

23. The method of claim 22, wherein the biologically active molecules include at least one of anti-inflammatory molecules and anti-apoptotic drugs.
